(19) United States
(12) Patent Application Publication
(10) Pub. No.: US 2025/0281670 A1
(43) Pub. Date: Sep. 11, 2025

(54) 3D Printing Photo-Curing Composition Suitable for Skull Repair

(71) Applicant: Beijing Tiantan Hospital, Capital Medical University, Beijing (CN)

(72) Inventors: Yilong WANG, Beijing (CN); Yuchun GU, Beijing (CN); Weiqi CHEN, Beijing (CN); Bowen CHI, Beijing (CN); Lida WU, Beijing (CN); Yongjun WANG, Beijing (CN); Lingling JIANG, Beijing (CN)

(21) Appl. No.: 18/916,480

(22) Filed: Oct. 15, 2024

(30) Foreign Application Priority Data
Mar. 8, 2024 (CN) 202410267789.0

(51) Int. Cl. A61L 27/22; A61L 27/10; A61L 27/20; A61L 27/34; A61L 27/36; A61L 27/44; A61L 27/52; B33Y 70/00

(52) U.S. Cl. CPC A61L 27/222; A61L 27/10; A61L 27/20; A61L 27/227; A61L 27/34; A61L 27/365; A61L 27/446; A61L 27/52; B33Y 70/00; A61L 2300/214; A61L 2400/12

(57) ABSTRACT

Provided is a three dimensional (3D) printing photo-curing composition suitable for skull repair. The 3D printing photo-curing composition suitable for skull repair includes the following components: nanoclay; a photo-crosslinking hydrogel; at least one photoinitiator; and at least one photoresist.

### CROSS REFERENCE TO RELATED APPLICATION

This patent application claims the benefit and priority of Chinese Patent Application No. 2024102677890 filed with the China National Intellectual Property Administration on Mar. 8, 2024, the disclosure of which is incorporated by reference herein in its entirety as part of the present application.

### TECHNICAL FIELD

The present disclosure relates to the technical field of medical materials, and in particular to a three dimensional (3D) printing photo-curing composition suitable for skull repair.

### BACKGROUND

Patching materials for skull defect repair include the patient's autogenous skull (which requires subcutaneous implantation or cryopreservation) as well as artificial grafts. The artificial grafts include polymethyl methacrylate (PMMA) patch, titanium plate, polyetheretherketone resin (PEEK) patch, and porous hydroxyapatite scaffold. However, each of the above patching materials has inherent limitations. For example, autogenous bone regrafting may cause self-absorption of a bone flap, resulting in repair failure; the titanium plate is a non-physiological heat conductor, which could interfere with imaging of imaging examinations and may increase the incidence of soft tissue atrophy; and a PEEK cranial bone flap has a relatively high cost, and shows rigidity and elasticity that do not fully match the mechanical properties of natural bone, leading to the risk of postoperative displacement. Moreover, the above three types of skull patches serve as physical scaffolds to protect intracranial tissue structures, but could not be used for skull repair to promote the reconstruction of an immune microenvironment.

In order to make the skull repair patch more closely fit the shape and size of the skull defect, 3D printing may be adopted to allow adaptive printing based on the scan data of a defected site. Among the currently commonly-used 3D printing methods, micro-extrusion bio-3D printing has become a reliable process for 3D printing of bone repair scaffolds due to its simple operation, high efficiency, and relatively low cost. However, since the printing process generally involves organic solvents or high temperatures, the biological activity of the scaffold may be lost. Moreover, the printing process also shows low precision and could easily lead to relatively large pore size in a printed product, which is not conducive to cell deposition and adhesion.

### SUMMARY

In order to solve the above problems, the present disclosure provides a three dimensional (3D) printing photo-curing composition suitable for skull repair. In the present disclosure, methacrylated gelatin (GelMA) and nanoclay are used as printing materials, which have desirable photo-crosslinking properties, high bio-compatibility, and mechanical strength that meets the supporting function. Furthermore, a 3D printing photo-curing process shows higher printing accuracy, while a printed product exhibits smaller pore size, which is conducive to cell adhesion, proliferation, and differentiation.

Additional features and advantages of the present disclosure will be set forth in part in the specification below, and in part will be apparent from the specification, or may be learned by practice of the present disclosure. The objects and other advantages of the present disclosure will be realized and obtained by means of elements and combinations particularly pointed out in the claims appended to the specification.

In order to achieve the above advantages, the present disclosure provides a 3D printing photo-curing composition suitable for skull repair, including the following components: nanoclay, a photo-crosslinking hydrogel, at least one photoinitiator, and at least one photoresist.

Furthermore, in order to further improve mechanical properties and cross-linking molding performance of the photo-curing composition while ensuring high bio-compatibility, the present disclosure further limits a weight ratio of the components of the photo-curing composition. In some embodiments, the photo-curing composition includes the following components based on a total weight of the 3D printing photo-curing composition: 1 wt % to 4 wt % of the nanoclay; 5 wt % to 20 wt % of the photo-crosslinking hydrogel; 0.5 wt % to 5 wt % of the at least one photoinitiator; and 0.001 wt % to 3 wt % of the at least one photoresist.

In order to obtain faster gel formation speed and better mechanical properties, in some embodiments, the photo-crosslinking hydrogel is at least one selected from the group consisting of methacrylated gelatin (GelMA), methacrylated sodium alginate (AlgMA), and methacrylated hyaluronic acid (HAMA). In some embodiments, the photo-crosslinking hydrogel is the GelMA. The GelMAis injectable, gels quickly, has desirable mechanical properties and bio-compatibility, and is suitable for customized bio-printing.

In order to obtain the mechanical strength of the photo-crosslinking hydrogel in the photo-curing composition without affecting temperature-sensitive properties of the hydrogel, in some embodiments, the nanoclay is at least one selected from the group consisting of hydroxyapatite, tricalcium phosphate, and lithium magnesium silicate. In some embodiments, the nanoclay is the lithium magnesium silicate. The lithium magnesium silicate has a nano-scale 3D structure, shows excellent physical and chemical properties as well as bio-compatibility, exhibits certain antibacterial properties, and could promote cell proliferation and differentiation.

In order to make the photo-curing composition have strong compression resistance such that it could be close to the mechanical strength of a human skull, and to solve a problem that the photo-curing composition prepared by the photo-crosslinking hydrogel may absorb water and swell in the skull, in some embodiments, the photo-curing composition further includes tea polyphenol. The photo-crosslinking hydrogel combines with the tea polyphenol in the form of hydrogen bonds, which could better absorb the tea polyphenol, thereby improving stability, mechanical properties, and antibacterial and antioxidant activity of the photo-curing composition.

In order to take into account the antibacterial effect and mechanical properties of the photo-crosslinking hydrogel combined with the tea polyphenol, in some embodiments, the tea polyphenol accounts for 0.1 wt % to 22 wt % of the total weight of the 3D printing photo-curing composition.

In some embodiments, the photoinitiator is at least one selected from the group consisting of phenyl ketone, phenyl dimethylketone, phenyl trimethylketone, benzoin ether, benzoin bis-ether, α-hydroxy-alkylphenone, and α-alkoxy-alkylphenone.

In some embodiments, the photoresist is at least one selected from the group consisting of Sudan Orange G, Sudan I, and Tinuvin 171.

In order to enable the photo-curing composition to improve differentiation of bone cells and blood vessels and reconstruction of the neuroimmune environment, in some embodiments, the photo-curing composition further includes a chemokine. The chemokine could enter the skull environment along with the photo-curing composition to promote cell differentiation and neuroimmune environment reconstruction during the skull repair.

In some embodiments, the chemokine is at least one selected from the group consisting of a ciliary neurotrophic factor (CNTF) neural factor, a vascular endothelial growth factor (VEGF) vascular factor, and a bone morphogenetic protein 2 (BMP-2) bone repair factor. The CNTF neural factor could promote the synthesis of neurotransmitters and the axonal growth of certain neuron groups in the nervous system, and could also be expressed in bone surface cells.

The VEGF vascular factor has effects such as promoting increased vascular permeability, extracellular matrix degeneration, vascular endothelial cell migration, proliferation, and blood vessel formation. The BMP-2 bone repair factor could stimulate DNA synthesis and cell replication, thereby promoting the directional differentiation of mesenchymal cells into osteoblasts. The BMP-2 bone repair factor is also a main factor inducing bone and cartilage formation in vivo, and is expressed during limb growth, endochondral ossification, early fracture, and cartilage repair, and plays an important role in embryonic development and regeneration repair of bones. Further, in order to enable the photo-curing composition to better promote the repair and differentiation of bone cells and the generation of peripheral blood vessels in skull defect after entering the skull, in some embodiments, the chemokine is composed of the CNTF neural factor, the VEGF vascular factor, and the BMP-2 bone repair factor, which work synergistically in the skull to promote skull repair.

Some embodiments of the present disclosure have the following beneficial effects:

(1) In the present disclosure, the photo-crosslinking hydrogel mainly composed of organic components and the nanoclay mainly composed of inorganic components are used as main raw materials of photo-curing printing ink, thus satisfying special material requirements of a 3D printing photo-curing process. Furthermore, the 3D printing photo-curing process shows higher printing accuracy, while a printed product exhibits smaller pore size, which is conducive to cell adhesion, proliferation, and differentiation.

(2) In the present disclosure, tea polyphenol is added to the photo-curing composition. The tea polyphenol could solve a problem that the photo-curing composition prepared by the photo-crosslinking hydrogel may absorb water and swell in the skull. The photo-crosslinking hydrogel combines with the tea polyphenol in the form of hydrogen bonds, which could better absorb the tea polyphenol, thereby improving mechanical properties, and antibacterial and antioxidant activity of the photo-curing composition.

### BRIEF DESCRIPTION OF THE DRAWINGS

To describe the technical solutions in the embodiments of the present disclosure more clearly, the following briefly describes the drawings required for describing the examples of the present disclosure. Apparently, the drawings in the following description show merely some embodiments of the present disclosure, and those of ordinary skill in the art may still derive other drawings from these drawings without creative efforts.

FIG. 1 shows an appearance diagram of the printed sample in Example 1 of the present disclosure;

FIG. 2 shows a mechanical property test of the printed sample in Example 1 of the present disclosure;

FIG. 3 shows long-term swelling test results of the printed sample in Example 1 of the present disclosure;

FIGS. 4A-4D show the staining of live and dead cells on the printed sample in Example 1 of the present disclosure under Live, dead, TD, and merge light source channels of a cell transplantation test, respectively;

FIG. 5 shows micro-CT results of the effectiveness of skull defect repair in animal experiments on the printed sample in Example 1 of the present disclosure;

FIG. 6 shows the Masson staining effect of the skull in animal experiments on the printed sample in Example 1 of the present disclosure;

FIG. 7 shows a schematic diagram of the appearance of the printed sample in Comparative Example 1 of the present disclosure;

FIG. 8 shows a mechanical property test of the printed sample in Comparative Example 1 of the present disclosure;

FIG. 9 shows long-term swelling test results of the printed sample in Comparative Example 1 of the present disclosure;

FIGS. 10A-10D show the staining of live and dead cells on the printed sample in Comparative Example 1 of the present disclosure under Live, dead, TD, and merge light source channels of a cell transplantation test, respectively;

FIG. 11 shows micro-CT results of the effectiveness of skull defect repair in animal experiments of the printed sample in Comparative Example 1 of the present disclosure;

FIG. 12 shows the Masson staining effect of the skull in animal experiments on the printed sample in Comparative Example 1 of the present disclosure;

FIG. 13 shows a schematic diagram of an appearance of the printed sample in Comparative Example 2 of the present disclosure;

FIG. 14 shows a mechanical property test of the printed sample in Comparative Example 2 of the present disclosure;

FIG. 15 shows long-term swelling test results of the printed sample in Comparative Example 2 of the present disclosure;

FIGS. 16A-16D show the staining of live and dead cells on the printed sample in Comparative Example 2 of the present disclosure under Live, dead, TD, and merge light source channels of a cell transplantation test, respectively;

FIG. 17 shows micro-CT results of the effectiveness of skull defect repair in animal experiments on the printed sample in Comparative Example 2 of the present disclosure; and

FIG. 18 shows the Masson staining effect of the skull in animal experiments on the printed sample in Comparative Example 2 of the present disclosure.

### DETAILED DESCRIPTION OF THE EMBODIMENTS

The photo-curing composition according to the present disclosure will be described in more detail with reference to the following embodiments. Apparently, the described embodiments are some rather than all of the embodiments of the present disclosure. All other embodiments obtained by those of ordinary skill in the art based on the embodiments of the present disclosure without creative efforts should fall within the protection scope of the present disclosure.

In the description of the embodiments of the present disclosure, it should be noted that all ranges disclosed in the present disclosure will be understood to encompass any and all sub-ranges included therein. For example, a stated range of “2.0 to 13.0” should be deemed to include any and all sub-ranges that begin with a minimum value of 2.0 or greater and end with a maximum value of 13.0 or less, for example, 2.0 to 6.2, or 3.5 to 10.0, or 5.2 to 7.9. Also, all ranges disclosed herein are deemed to include endpoints of the ranges unless expressly stated otherwise. For example, the range “between 4 and 6” or “4 to 6” or “4-6” should generally be considered to include endpoints 4 and 6.

Likewise, it is to be understood that phrases and terminology used in the present disclosure are for the purpose of description and should not be regarded as limiting. The use of “comprising”, “,including”, or “having” and variations thereof in the disclosure is intended to include the items listed thereafter and their equivalents as well as additional items in an open-ended manner.

As used herein in the specification and claims, the phrase “at least one”, with reference to a list of one or more elements, should be understood to mean at least one element selected from any one or more elements in the list of elements. However, at least one of every element specifically enumerated in the element list is not necessarily included, and any combination of elements in the element list is not excluded.

The term “3D printing” and the like generally describe a variety of solid free-form manufacturing techniques for fabricating three-dimensional articles or objects by: stereolithography, selective deposition, jetting, fused deposition molding, multi-jet molding, digital light processing, gel deposition, as well as other additive manufacturing technologies known in the art or known in the future that use building materials or inks to create three-dimensional objects.

The primary meaning of the term “bio-compatibility” when describing hydrogels in the present disclosure is to provide positive and controllable effects on biological and functional structures, including interactions with endogenous tissues or the immune system, supporting appropriate cellular activity, and promoting molecular or mechanical signaling systems. These effects are critical to the biological function of a graft.

The present disclosure provides a photo-curing composition, including: nanoclay, a photo-crosslinking hydrogel, at least one photoinitiator, at least one photoresist, tea polyphenol, and a chemokine.

### Nanoclay

In the present disclosure, the nanoclay may include nanoparticles of layered mineral silicates such as hydroxyapatite, tricalcium phosphate, and lithium magnesium silicate, may also include nanomaterials such as montmorillonite, bentonite, kaolinite, hectorite, and halloysite that have certain adsorption capabilities and are compatible with a variety of polymer systems, and may also include degradable materials such as calcium silicate, bioglass, akermanite, calcium akermanite, and diopside.

### Photo-Crosslinking Hydrogel

An important feature of biomaterials suitable for bio-printing is the ability to rapidly form in situ and ensure the existence of cells through various cross-linking methods, including physical methods, chemical methods, and photo-crosslinking methods. In the present disclosure, the photo-crosslinking hydrogel refers to a compound containing a photosensitive group that could form a three-dimensional network hydrogel through intramolecular or intermolecular cross-linking under the irradiation of ultraviolet light or visible light. There are two main types of common photo-crosslinking methods. One type is that under the irradiation of ultraviolet light, the photoinitiator undergoes a cleavage reaction to form active free radicals, and a polymer containing vinyl functional groups is initiated to undergo a series of rapid polymerization reactions to form a gel. The other type is that without additional photoinitiators, a polymer containing cross-linkable groups undergoes a cross-linking reaction to form a gel. Compared with other cross-linking methods, the photo-crosslinking reaction has mild conditions, fewer by-products, no need for toxic initiators, and a process of the cross-linking reaction could be controlled by simply controlling intensity, irradiation time, and irradiation range of light. In addition, the photo-crosslinking hydrogel could quickly encapsulate cells and growth factors in situ, thereby improving survival rate of cells. The presence of growth factors plays an important role in promoting cell proliferation and differentiation, tissue repair, and regeneration. When being synchronously photopolymerized and printed with cells into a three-dimensional hydrogel, the growth factors could improve the viability of the encapsulated cells.

In some embodiments of the present disclosure, the photo-crosslinking hydrogel includes at least one selected from the group consisting of GelMA, AlgMA, and HAMA. In some embodiments, the photo-crosslinking hydrogel is the GelMA. It should be noted that when using the GelMA as a photo-curing printing material, it is crucial to control the viscosity of the GelMA. A high viscosity of the photo-curing printing material could maintain fidelity of deposition in 3D printing, and a higher viscosity could usually be obtained by adjusting a concentration of a GelMA solution. However, an excessive concentration of the GelMA may lead to a dense polymer network, inevitably reducing cell activity. Therefore, in some embodiments of the present disclosure, the GelMA has a concentration of 5 wt % to 20 wt %, preferably 10 wt % to 15 wt %.

### Chemokine

In the present disclosure, the chemokine refers to a type of small cytokine or signaling protein secreted by cells. The chemokine has the ability to induce directional chemotaxis of nearby reaction cells, such that the human body could promote directional chemotaxis of immune cells when the human body defends and removes foreign bodies such as invading pathogens. The chemokine may include any one selected from the group consisting of a CNTF neural factor, a VEGF vascular factor, and a BMP-2 bone repair factor, or a mixture of two or more of the above chemokines. In some embodiments, the chemokine is a mixture of the CNTF neural factor, the VEGF vascular factor, and the BMP-2 bone repair factor.

### Photoinitiator

In the present disclosure, the photoinitiator refers to an agent capable of initiating photopolymerization. The photoinitiator may include any one selected from the group consisting of phenyl ketone, phenyl dimethylketone, phenyl trimethylketone, benzoin ether, benzoin bis-ether, α-hydroxy-alkylphenone, and α-alkoxy-alkylphenone, or a mixture of two or more of the above photoinitiators. In some embodiments, the photoinitiator further includes 2-naphthalenesulfonyl chloride and analogs thereof, methoxyacetophenone and analogs thereof, benzoin and analogs thereof, benzyl and analogs thereof, benzophenone, and benzyl dimethyl ketal and analogs thereof.

### Photoresist

In the present disclosure, the photoresist refers to an agent added to inhibit lateral photopolymerization during 3D printing. The photoresist may include Sudan Orange G, Sudan I, and Tinuvin 171.

The nanoclay is present in the photo-curing composition in the following weight percentages: 1 wt % to 4 wt %; 1 wt % to 3 wt %; 1 wt % to 2 wt %; 2 wt % to 4 wt %; about 3 wt %.

The photo-crosslinking hydrogel is present in the photo-curing composition in the following weight percentages: 5 wt % to 20 wt %; 8 wt % to 18 wt %; 10 wt % to 16 wt %; 12 wt % to 18 wt %; 13 wt % to 17 wt %; 15 wt % to 19 wt %; 16 wt % to 18 wt %; about 15%.

The photoinitiator is present in the photo-curing composition in the following weight percentages: 0.5 wt % to 5 wt %; 1 wt % to 5 wt %; 2 wt % to 4.5 wt %; 2.5 wt % to 4 wt %; 2.5 wt % to 3.5%; 2.5 wt % to 3 wt %; about 3.5 wt %.

The photoresist is present in the photo-curing composition in the following weight percentages: 0.001 wt % to 3 wt %; 0.3 wt % to 2.8 wt %; 0.5 wt % to 2.6 wt %; 1.2 wt % to 2.4 wt %; 1.5 wt % to 2.2 wt %; about 1.5 wt %.

The tea polyphenol is present in the photo-curing composition in the following weight percentages: 0.1 wt % to 22 wt %; 1 wt % to 20 wt %; 5 wt % to 18 wt %; 8 wt % to 16 wt %; 10 wt % to 15 wt %; 12 wt % to 14 wt %; about 11 wt %.

The chemokine is present in the photo-curing composition at a concentration of: the CNTF neural factor (0.1 g/mL to 100 g/mL), the VEGF vascular factor (1 g/mL to 100 g/mL), the BMP-2 bone repair factor (0.1 g/mL to 200 g/mL); the CNTF neural factor (30 g/mL to 80 g/mL), the VEGF vascular factor (30 g/mL to 80 g/mL), the BMP-2 bone repair factor (50 g/mL to 180 g/mL); the CNTF neural factor (30 g/mL to 80 g/mL), the VEGF vascular factor (20 g/mL to 80 g/mL), the BMP-2 bone repair factor (30 g/mL to 180 g/mL); the CNTF neural factor (35 g/mL to 60 g/mL), the VEGF vascular factor (38 g/mL to 80 g/mL), the BMP-2 bone repair factor (50 g/mL to 160 g/mL); the CNTF neural factor (about 50 g/mL), the VEGF vascular factor (about 50 g/mL), the BMP-2 bone repair factor (about 100 g/mL).

The present disclosure is further described below in conjunction with specific examples.

### Example 1

Example 1 provided a 3D printing photo-curing composition suitable for skull repair and a preparation method thereof, where the preparation method was conducted as follows:

Preparation of a nanoclay solution: 0.2006 g of nanoclay was weighed and then added to 10 mL of sterile purified water, and subjected to ultrasonic mixing for 3 min to be uniform to obtain the nanoclay solution with a concentration of 2%.

Preparation of the nanoclay solution combined with chemokines: a CNTF neural factor (0.1 g/mL)+a VEGF vascular factor (1 g/mL)+a BMP-2 bone repair factor (0.1 g/mL) were dissolved in the nanoclay solution, and stored at a temperature of 37° C. in the dark for later use.

Preparation of a printing photo-curing composition precursor: 0.7506 g of a GelMA freeze-dried sample was weighed and mixed with 5 mL of a prepared 2 wt % nanoclay solution combined with chemokines. A resulting mixture was shaken in a centrifuge at a rotation speed of 1,000 rpm for 15 min to obtain the printing photo-curing composition precursor with a GelMA concentration of 15%, and stored at a temperature of 37° C. in the dark for later use.

Preparation of the printing photo-curing composition: 1 mL of the printing photo-curing composition precursor, 60 μL of a 5 wt % photoinitiator, and 17 μL of a 3 wt % photoresist were mixed to be uniform to obtain a mixture, which was prepared for immediate use. At the same time, 0.5 mL of a tea polyphenol solution was prepared as an outer surface coating of a printed sample and added into an ink tank of a photo-curing printer. Printing was conducted using a projection photo-curing printer, with an exposure time of 6 s for each layer and a light intensity of 20 mW/cm² to obtain the photo-curing printed sample.

### Comparative Example 1

This comparative example was conducted similar to Example 1, except that the chemokine was not added. This comparative example provided a 3D printing photo-curing composition for skull repair and a preparation method thereof, where the preparation method was conducted as follows:

Preparation of a nanoclay solution: 0.2006 g of nanoclay was added to 10 mL of sterile purified water, and subjected to ultrasonic mixing for 3 min to be uniform to obtain the nanoclay solution with a concentration of 2%.

Preparation of a printing photo-curing composition precursor: 0.7506 g of a GelMA freeze-dried sample was weighed and mixed with 5 mL of a prepared 2 wt % nanoclay solution. A resulting mixture was shaken in a centrifuge at a rotation speed of 1,000 rpm for 15 min to obtain the printing photo-curing composition precursor with a GelMA concentration of 15%, and stored at a temperature of 37° C. in the dark for later use.

Preparation of the printing photo-curing composition: 1 mL of the printing photo-curing composition precursor, 60 μL of a 5 wt % photoinitiator, and 17 μL of a 3 wt % photoresist were mixed to be uniform to obtain a mixture, which was prepared for immediate use. At the same time, 0.5 mL of a tea polyphenol solution was prepared as an outer surface coating of a printed sample and added into an ink tank of a photo-curing printer. Printing was conducted using a projection photo-curing printer, with an exposure time of 6 s for each layer and a light intensity of 20 mW/cm² to obtain the photo-curing printed sample.

### Comparative Example 2

This comparative example was conducted similar to Example 1, except that the tea polyphenol was not added to a photo-curing composition as an outer surface coating of a printed sample. This comparative example provided a 3D printing photo-curing composition for skull repair and a preparation method thereof, where the preparation method was conducted as follows:

Preparation of a nanoclay solution: 0.2006 g of nanoclay was added to 10 mL of sterile purified water, and subjected to ultrasonic mixing for 3 min to be uniform to obtain the nanoclay solution with a concentration of 2%.

Preparation of the nanoclay solution combined with chemokines: a CNTF (0.1 g/mL) neural factor+a VEGF vascular factor (1 g/mL)+a BMP-2 bone repair factor (0.1 g/mL) were dissolved in the nanoclay solution, and stored at a temperature of 37° C. in the dark for later use.

Preparation of a printing photo-curing composition precursor: 0.7506 g of a GelMA freeze-dried sample was weighed and mixed with 5 mL of a prepared 2 wt % nanoclay solution combined with chemokines. A resulting mixture was shaken in a centrifuge at a rotation speed of 1,000 rpm for 15 min to obtain the printing photo-curing composition precursor with a GelMA concentration of 15%, and stored at a temperature of 37° C. in the dark for later use.

Preparation of the printing photo-curing composition: 1 mL of the printing photo-curing composition precursor, 60 μL of a 5 wt % photoinitiator, and 17 μL of a 3 wt % photoresist were mixed to be uniform to obtain a mixture, which was prepared for immediate use. The mixture was added into an ink tank of a photo-curing printer. Printing was conducted using a projection photo-curing printer, with an exposure time of 6 s for each layer and a light intensity of 20 mW/cm² to obtain the photo-curing printed sample.

The advantages of Example 1 in terms of printing effect were demonstrated through the following tests.
I. Appearance test: visual inspection;
II. Printing effect test: observation was conducted to see whether the appearance of a sample printed using the same program was consistent with the program settings;
III. Mechanical properties test:
3.1 Instruments and equipment: Universal testing machine.
3.2 Experimental method
3.2.1. The universal testing machine was turned on and preheated for five minutes.
3.2.2. The software was opened, the universal testing machine was connected, and the testing machine was started. A method was set as compression method. The sample size-compression speed-polar direction were adjusted, and the method was saved.
3. The sample was placed in the middle of a clamp, the upper clamp was lowered to 1 mm from an upper surface of a sample using a manual control box. Force and displacement were reset to zero on the software, and the testing was started to obtain the mechanical performance diagram of the sample.
3.2.4. A curve was observed, the experiment was stopped after the material was broken. The clamp was lifted using the manual control box, surfaces of a broken sample and the clamp were cleaned with lint-free cloth and alcohol, and the curve graph and experimental data were saved to a computer.
3.2.5. After the experiment, the software and the universal testing machine were turned off in sequence.
IV. Long-term swelling performance test
4.1 Experimental reagent: PBS solution
4.2 Experimental method

A newly prepared gel was weighed as G1 and placed in a 12-well plate. 1 mL of sterile water was added to each well to fully immerse the printed sample in Examples. After incubation at a temperature of 37° C., the gel sample was taken out at 1st, 3rd, 7th, 14th, and 21st days separately, and a long-term swelling rate of the gel sample was calculated. The gel sample was dried with dust-free paper and placed on a glass slide to weigh to obtain a wet weight G2. The swelling rate of the gel sample was calculated according to the formula and a curve was plotted to obtain a swelling test results diagram.

$$Swelling rate = \frac{G 2 - G 1}{G 1} \times 100 \%$$

V. Cell bio-compatibility test:
5.1 Principle

Calcein acetoxymethyl ester (Calcein-AM) is a cell staining reagent for fluorescent labeling of living cells, and emits green fluorescence (Ex=490 nm, Em=515 nm). The Calcein-AM introduces an acetyl methoxymethyl ester (AM) group on the basis of traditional Calcein to increase hydrophobicity, allowing the Calcein-AM to easily penetrate living cell membranes. Once inside the cell, the Calcein-AM (which itself does not fluoresce) is cleaved by intracellular esterases to form the membrane-impermeable polar molecule Calcein, and then retained in the cell and emits strong green fluorescence. Propidiumiodide (PI) can not pass through the cell membranes of the living cells, but can only pass through the disordered area of dead cell membranes to reach nucleus, and is embedded in the cell's DNA double helix to produce red fluorescence (Ex=535 nm, Em=617 nm), such that the PI only stains dead cells.
5.2 Instruments and equipment

| Instrument Name | Specification or model | Manufacturer |
|---|---|---|
| Laser scanning confocal | 37XB | Shanghai Optical |
| microscope | | Instruments, China |
| Single-channel manual | 100 μL to 1000 μL | ThermoFisher |
| adjustable pipette | | |
| Single-channel manual | 20 μL to 200 μL | ThermoFisher |
| adjustable pipette | | |
| Centrifuge | Sorvall ST1 Plus | ThermoFisher |
| Cell counter | AP-0650010 | MARIENFELD |

5.2.1 Reagents

| Reagent name | Cat. No. | Manufacturer |
|---|---|---|
| PBS | AH30026713 | Hyclone |
| Calcein-AM/PI | CA1630 | Solarbio |
| Assaybuffer | EPX-11110-000 | Thermo |

5.2.2 Consumables

| Consumables | Cat. No. | Manufacturer |
|---|---|---|
| 1.5 mL EP tube | 509-GRD-Q | QSP |
| 50 mL centrifuge tube | 339652 | ThermoNunc |
| 15 mL centrifuge tube | 339650 | ThermoNunc |
| 24-well culture plate | 142475 | ThermoNunc |

5.3 Experimental method
5.3.1 Cell seeding on a printed sample.
5.3.1.1 Three specimens were selected from each group of freeze-dried printed samples and sterilized with ultraviolet irradiation for 30 min.
5.3.1.2 Cells in a T75 culture flask were digested and counted. A concentration of a cell suspension was adjusted to 4×10⁴ cells/50 μL.
5.3.1.3 50 μL of the cell suspension was added dropwise to the center of each printed sample, incubated in a CO₂ incubator at 37° C. 150 μL of the cell suspension was added every 30 min to keep the printed sample moist. After incubation for 1 h, 800 μL of fresh medium was added.
5.3.1.4 On the 1st, 7th, and 14th days after seeding cells, live and dead cells were stained and photographed under a confocal microscope; undetected specimens should be replaced with medium every 2 d to 3 d.
5.3.2 Live and dead cell staining steps
5.3.2.1 10×AssayBuffer was taken out from the low-temperature refrigerator, dissolved at room temperature, and 1.5 mL of dissolved 10×AssayBuffer was added to 13.5 mL of sterilized water to obtain 15 mL of 1×AssayBuffer.
5.3.2.2. Preparation of 1× staining working solution: the cryopreserved Calcein-AM solution and PI solution stored at a relatively low temperature were returned to room temperature for 30 min.
5.3.2.3 0.75 μL of Calcein-AM solution and 2.25 μL of PI solution were added to 1.5 mL of 1×AssayBuffer, and mixed thoroughly. A cell-loaded printed sample was removed from the 12-well plate and placed to a new 12-well plate. The cell-loaded printed sample was washed 2 times with 1×AssayBuffer. 500 μL of the staining working solution was added to each well and incubated at a temperature of 37° C. for 30 min.
5.3.2.4 The staining working solution was removed, then added with 1×AssayBuffer and washed 2 times, and the original medium was added to the well plate.
5.3.3 Photographing
5.3.3.1 An FV3000 system was started according to the opening sequence of the laser confocal instrument.

A cell sample was found through an eyepiece. A button “Switch Objective” above TPC interface was clicked to switch objective lens. A DIA button was clicked, and a Z-axis position was adjusted to find a focal plane of the cell sample.
5.3.3.2 A detection channel, a green fluorescence channel (Ex=490 nm, Em=515 nm), and a red fluorescence channel (Ex=535 nm, Em=617 nm) were set in a [PMTsetting] tool window.
5.3.3.3 A button “Live” under [LIVE] tool window was clicked, the focal plane effect was adjusted according to preview image effect, and appropriate laser intensity (%), HV value (V), Gain (X) and other parameters were set.
5.3.3.4 A button “File” in [Acquire] tool window was clicked, a folder was selected to save the image, a file name for the image was entered, and a button “LSM Start” was clicked to start image acquisition.
5.3.3.5 Under a fluorescence microscope, living cells (yellow-green fluorescence) were detected with an excitation filter of 490±10 nm and dead cells (red fluorescence) were detected with an emission filter of 545 nm simultaneously.
5.3.3.6 Photographing was conducted on the live cell filter through four different light source channels (Live, dead, TD (i.e. no fluorescence), and merge) to obtain the staining images of the live and dead cells.
VI. In vivo experiment on skull defect repair with hydrogel
6.1 microCT detection of osteogenesis efficiency:
6.1.1 Experimental materials:
1. Experimental animals: SD rats, male, 6 Weeks to 8 Weeks
2. Print sample
3. Surgical instruments
4. Anesthetics
5. microCT scanner
6.1.2 Experimental steps:
1) Preparation before animal surgery: in accordance with experimental animal ethics and operating procedures, all experimental animals and equipment required for surgery were prepared in advance. The print sample was prepared in advance and injected into the skull defect according to the experimental design.
2) Surgical operation: the experimental animals were anesthetized and fixed on an operating table. A surgical site was prepared by local disinfection and shaving. Preparation of a skull defect model: a standard size defect was created in the rat skull using the surgical instruments. The pre-prepared hydrogel was filled into the skull defect site. The wound was sutured and subsequent treatments such as disinfection and pain relief medication were administered.
3) Postoperative management: the experimental animals were restored to an anesthetized awake state and placed in a comfortable environment. The recovery of experimental animals was observed.
4) End of experiment and sampling: the experiment was terminated at a predetermined time point (for example, 2 months after surgery). The animals were sacrificed under CO₂ conditions and the entire skull was removed. The experimental animals were scanned using a microCT scanner to obtain a micro-CT results diagram of the effectiveness of skull defect repair in the sample animal experiment, thus evaluating the skull defect repair situation.
6.2 In vivo effectiveness of hydrogel detected by Masson-stained sections
6.2.1
4% Paraformaldehyde (PFA)
Decalcification, dehydration, transparency, and embedding reagents
Microtome and tissue slicer
Masson staining kit
Optical microscopes and digital image acquisition systems
6.2.2
1) End of experiment and sampling:

The skull samples in 6.1 were photographed and fixated with 4% PFA for 12 h to maintain tissue morphology. The skull samples were decalcified, dehydrated, transparent, and embedded.

2) Slicing and staining: the skull samples were cut into 5 μm to 20 μm slices using a microtome and tissue microtome, and fixed on glass slides. Masson staining was conducted on the slices using a Masson staining kit to obtain Masson staining effect diagrams of the sample, thus observing the tissue structure and distribution of the hydrogel. Masson-stained slices were observed using optical microscopy to evaluate tissue structure, inflammatory response, and bio-compatibility of the hydrogel.

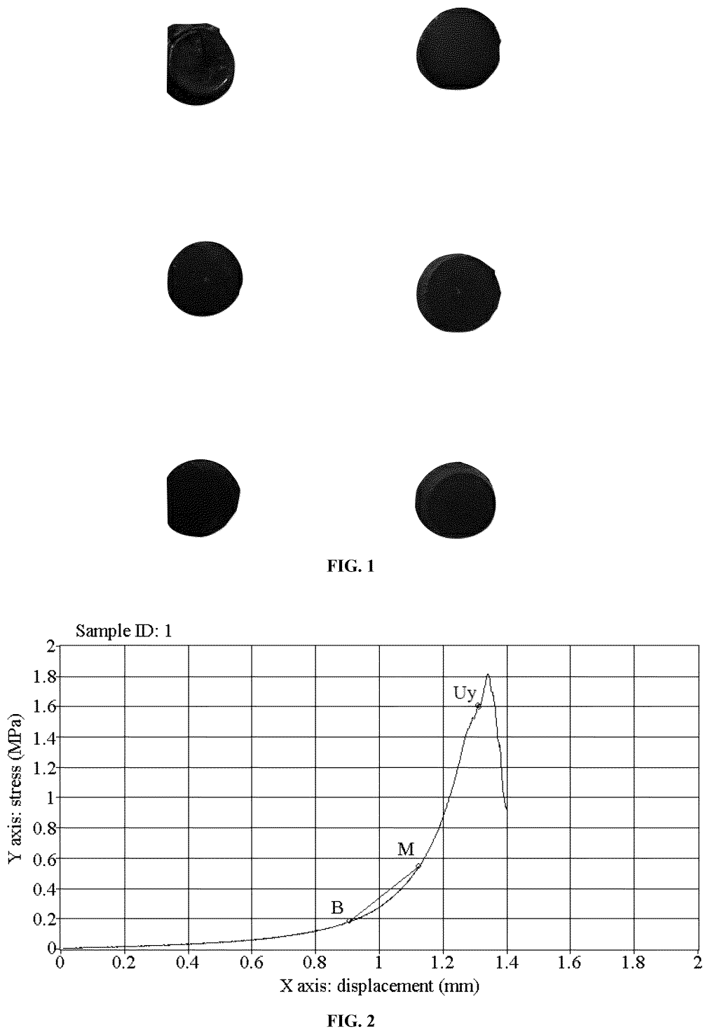

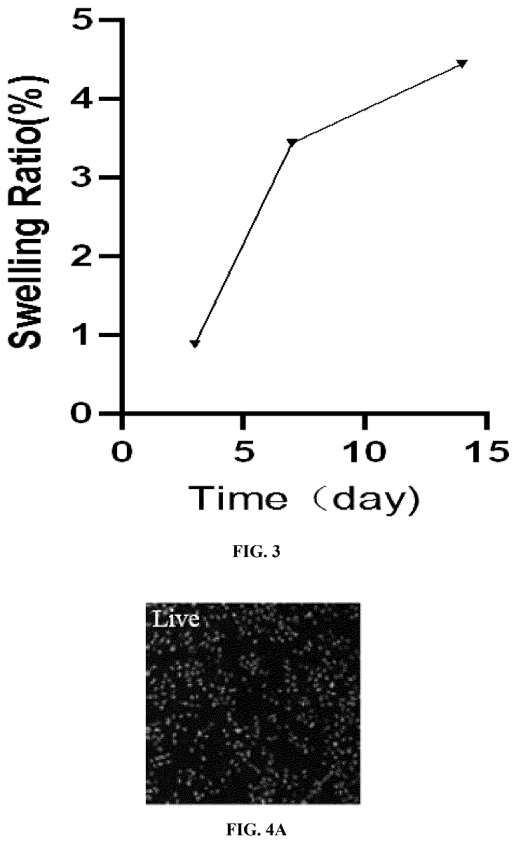

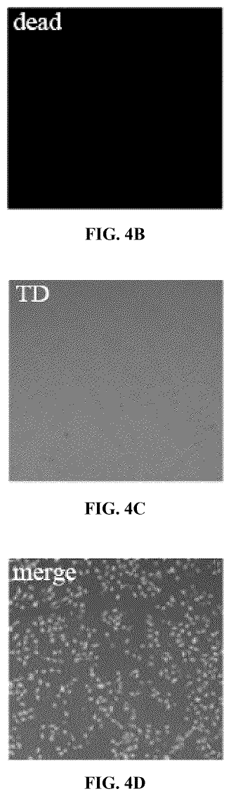

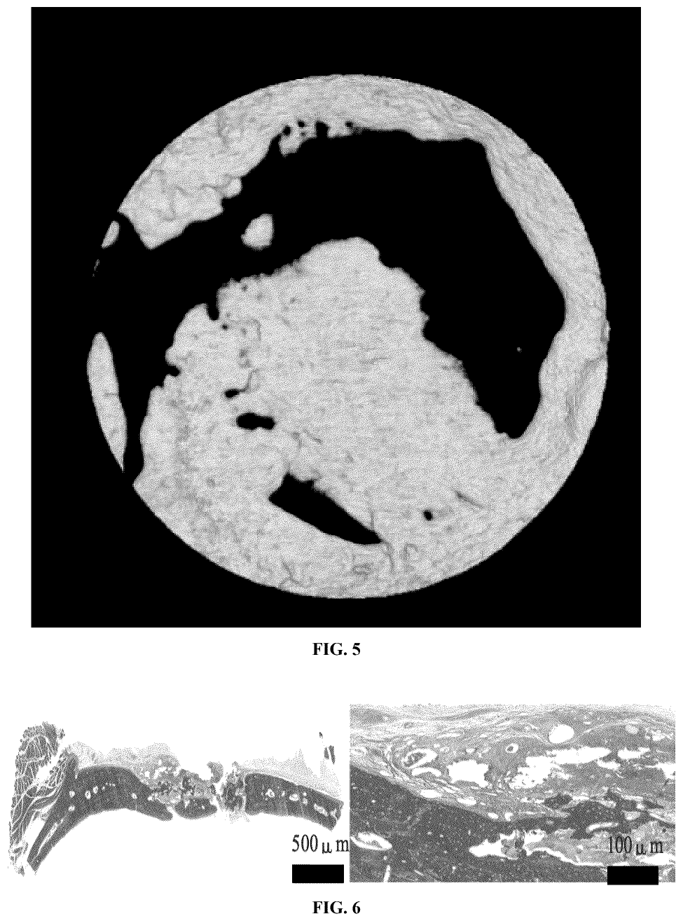

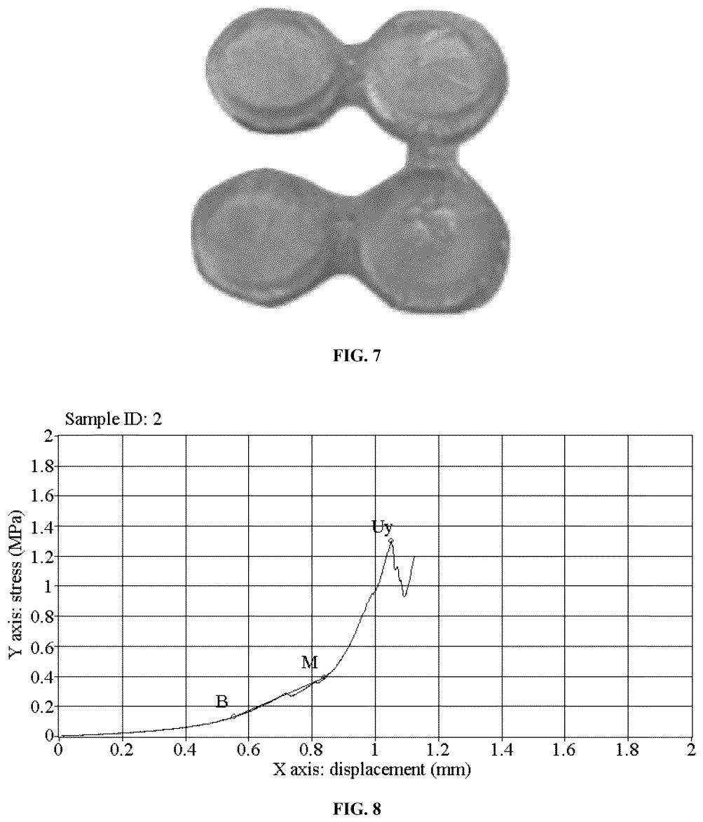

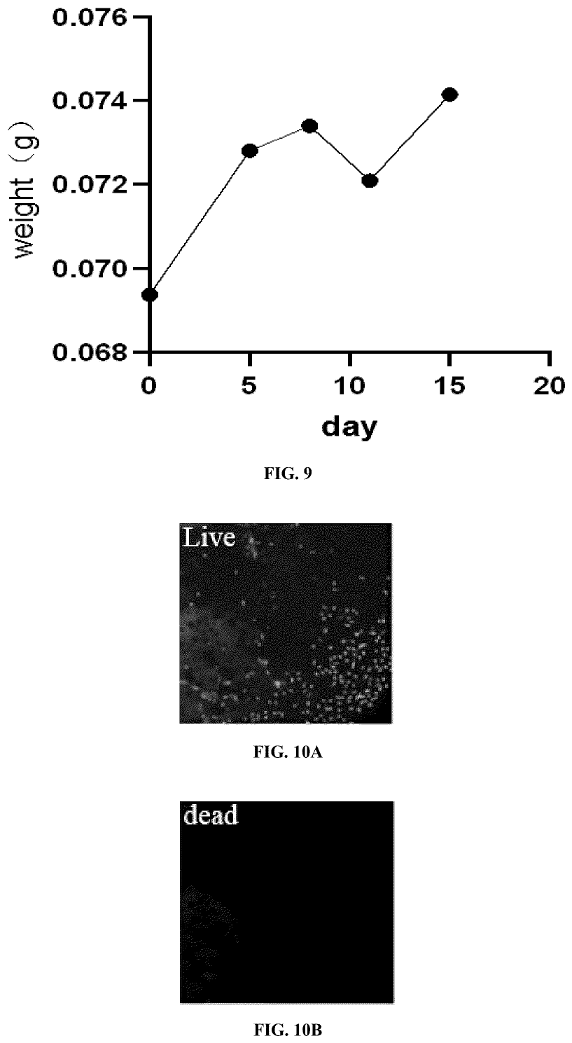

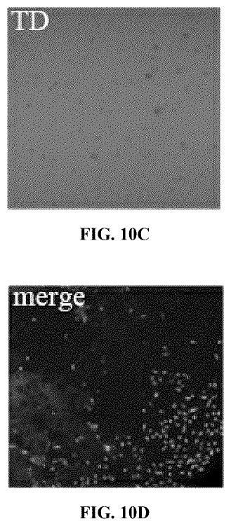

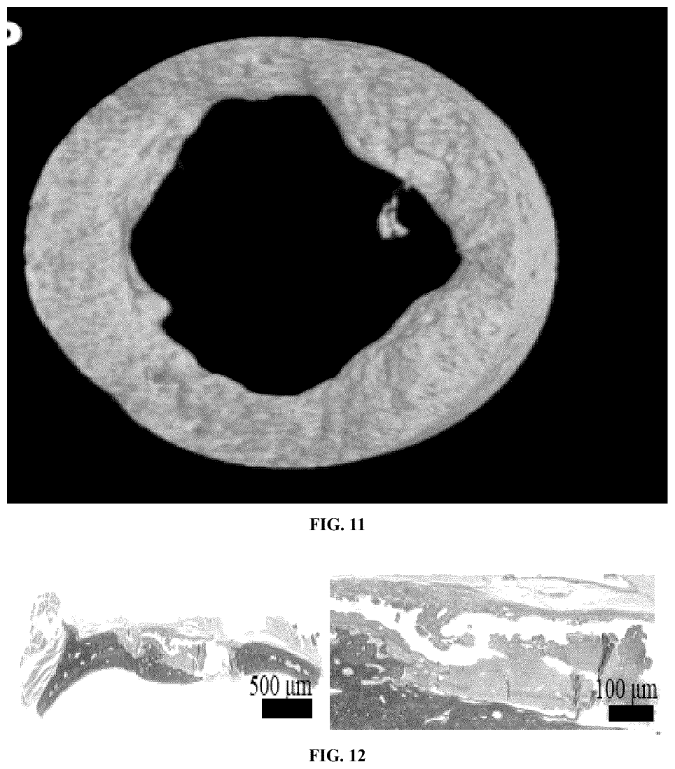

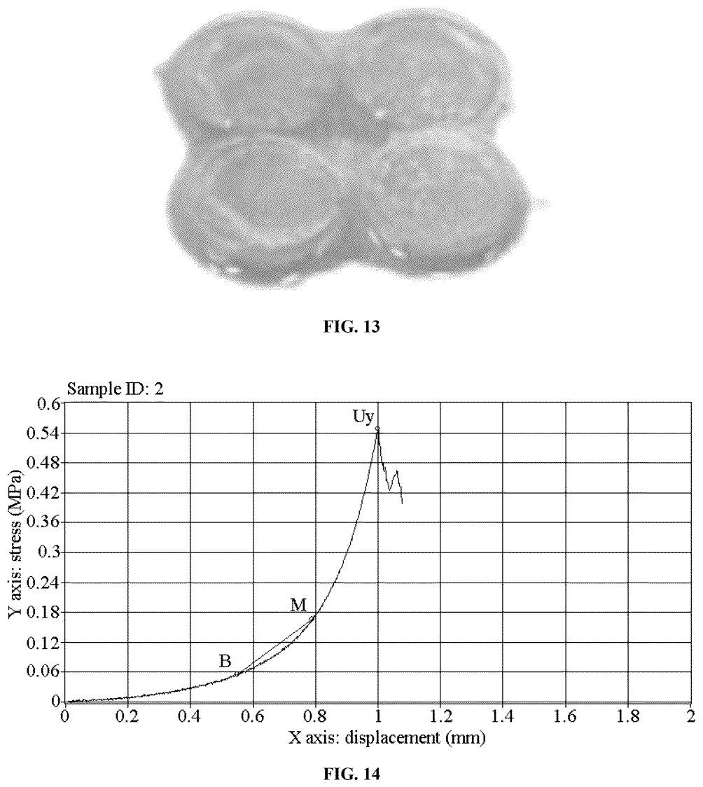

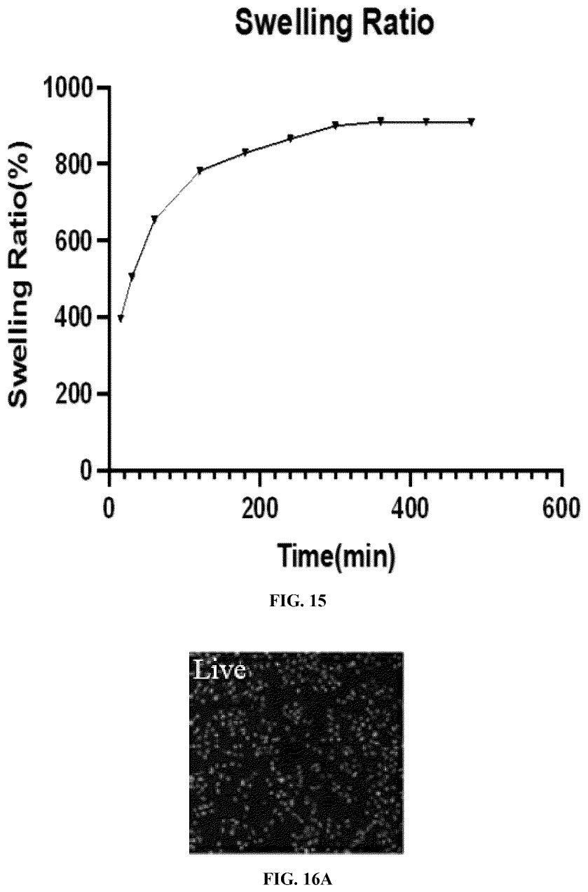

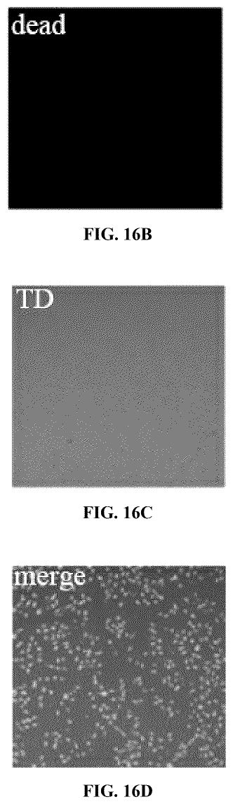

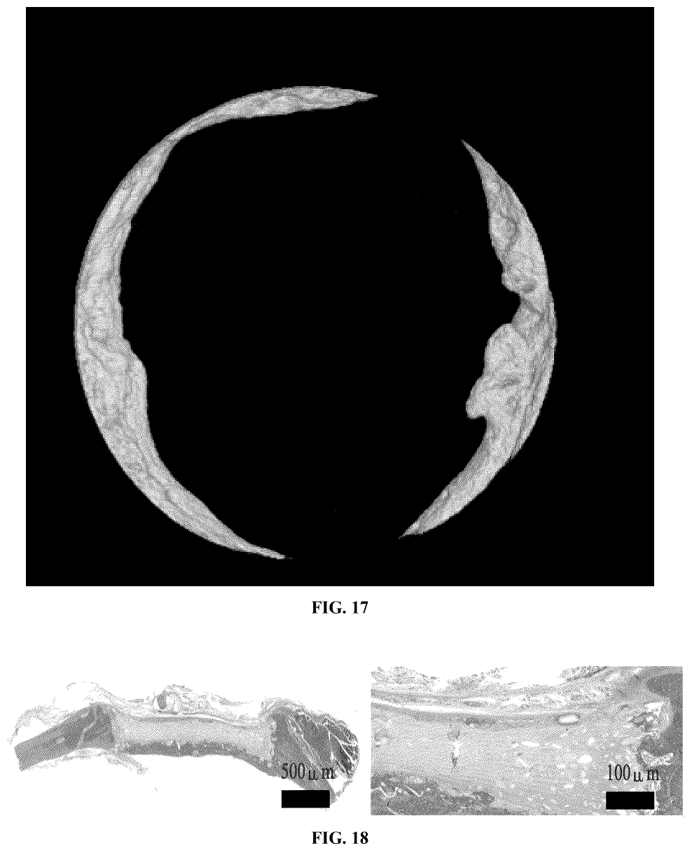

Test results are as follows:

| Test item | Example 1 | Comparative Example 1 | Comparative Example 2 |
|---|---|---|---|
| Appearance | Transparent or milky | Transparent or milky | Transparent or milky |
| | white liquid, liquid | white liquid, liquid state | white liquid, liquid state |
| | state at a temperature of | at a temperature of 37° C., | at a temperature of 37° C. |
| | 37° C., milky white | milky white semi-solid | that was relatively |
| | semi-solid state at a | state at a temperature of | viscous, milky white |
| | temperature of 4° C. | 4° C. After adding 5 wt % | semi-solid state at a |
| | After adding 5 wt % | photoinitiator and 3 wt % | temperature of 4° C. |
| | photoinitiator and 3 | photoresist to the bioink | After adding 5 wt % |
| | wt % photoresist to the | precursor, the printing | photoinitiator and 3 wt % |
| | bioink precursor, the | ink became a yellow | photoresist to the bioink |
| | printing ink became a | transparent liquid | precursor, the printing |
| | yellow transparent | | ink became a yellow |
| | liquid. | | transparent liquid |
| Printing effect | After printing, the | After printing, the | After printing Clear |
| | sample was consistent | sample was consistent | sample was consistent |
| | with the printing | with the printing | with the printing |
| | settings, completely | settings, completely | settings, completely |
| | cured, and had clear | cured, the printed sample | cured, the printed sample |
| | boundaries. The | was intact and had | was relatively intact and |
| | appearance of the | relatively clear | had relatively clear |
| | sample after soaking in | boundaries. The | boundaries, showing |
| | tea polyphenol was tan, | appearance of the sample | transparent, as shown in |
| | as shown in FIG. 1. | after soaking in tea | FIG. 13. |
| | | polyphenol was tan, as | |
| | | shown in FIG. 7. | |
| Mechanical | The sample size was | The sample size was | The sample size was |
| properties | 8*8*2 mm, the | 8*8*2 mm, the | 8*8*2 mm, the |
| | compression | compression | compression |
| | performance was 1.8 | performance was 1.253 | performance was 0.548 |
| | MPa, and the | MPa, and the mechanical | MPa, and the mechanical |
| | mechanical | performance test is | performance test is |
| | performance test is | shown in FIG. 8. | shown in FIG. 14. |
| | shown in FIG. 2. | | |
| Bio-compatibility | Referring to FIGS. | Referring to FIGS. | Referring to FIGS. |
| | 4A-4D (relatively | 10A-10D (ordinary cell | 16A-16D (ordinary cell |
| | desirable cell growth | growth status) | growth status) |
| | status) | | |
| In-body | As shown in FIG. 5, a | As shown in FIG. 11, a | As shown in FIG. 17, a |
| evaluation | large amount of new | part of new bone | part of new bone |
| | bone production is seen | production is seen in CT. | production is seen in CT. |
| | in CT. As shown in | As shown in FIG. 12, a | As shown in FIG. 18, a |
| | FIG. 6, a large number | part of new collagen | part of new collagen |
| | of new collagen blood | blood vessels are seen in | blood vessels are seen in |
| | vessels are seen in | Masson staining. During | Masson staining. During |
| | Masson staining. | the observation period, | the observation period, |
| | During the observation | there were no | there were no |
| | period, there were no | abnormalities in the | abnormalities in the |
| | abnormalities in the | weight and living | weight and living |
| | weight and living | conditions of the rats. | conditions of the rats. |
| | conditions of the rats. | | |

Comparison of the above test results showed that adding GelMA and tea polyphenol could give a printed sample better mechanical properties and bio-compatibility.

The above embodiments are merely intended to explain the technical solutions of the present disclosure, rather than to limit the present disclosure. Although the present disclosure is described in detail in conjunction with the above embodiments, those of ordinary skill in the art should understand that they can still modify the technical solutions described in the above embodiments or make equivalent substitutions for some technical features therein. However, these modifications or substitutions do not make the essence of the corresponding technical solutions without departing from the spirit of the technical solutions of various embodiments of the present disclosure, and should fall within the scope of the present disclosure.

What is claimed is:

1. A three dimensional (3D) printing photo-curing composition suitable for skull repair, comprising the following components:
nanoclay;
a photo-crosslinking hydrogel;
at least one photoinitiator; and
at least one photoresist.

2. The 3D printing photo-curing composition suitable for skull repair of claim 1, comprising the following components based on a total weight of the 3D printing photo-curing composition:
1 wt % to 4 wt % of the nanoclay;
5 wt % to 20 wt % of the photo-crosslinking hydrogel;
0.5 wt % to 5 wt % of the at least one photoinitiator; and
0.001 wt % to 3 wt % of the at least one photoresist.

3. The 3D printing photo-curing composition suitable for skull repair of claim 1, wherein the photo-crosslinking hydrogel is at least one selected from the group consisting of methacrylated gelatin (GelMA), methacrylated sodium alginate (AlgMA), and methacrylated hyaluronic acid (HAMA).

4. The 3D printing photo-curing composition suitable for skull repair of claim 1, wherein the nanoclay is at least one selected from the group consisting of hydroxyapatite, tricalcium phosphate, and lithium magnesium silicate.

5. The 3D printing photo-curing composition suitable for skull repair of claim 1, further comprising tea polyphenol.

6. The 3D printing photo-curing composition suitable for skull repair of claim 5, wherein the tea polyphenol accounts for 0.1 wt % to 22 wt % of a total weight of the 3D printing photo-curing composition.

7. The 3D printing photo-curing composition suitable for skull repair of claim 1, wherein the photoinitiator is at least one selected from the group consisting of phenyl ketone, phenyl dimethylketone, phenyl trimethylketone, benzoin ether, benzoin bis-ether, α-hydroxy-alkylphenone, and α-alkoxy-alkylphenone.

8. The 3D printing photo-curing composition suitable for skull repair of claim 1, wherein the photoresist is at least one selected from the group consisting of Sudan Orange G, Sudan I, and Tinuvin 171.

9. The 3D printing photo-curing composition suitable for skull repair of claim 1, further comprising a chemokine.

10. The 3D printing photo-curing composition suitable for skull repair of claim 9, wherein the chemokine is at least one selected from the group consisting of a ciliary neurotrophic factor (CNTF) neural factor, a vascular endothelial growth factor (VEGF) vascular factor, and a bone morphogenetic protein 2 (BMP-2) bone repair factor.

11. The 3D printing photo-curing composition suitable for skull repair of claim 2, wherein the photo-crosslinking hydrogel is at least one selected from the group consisting of methacrylated gelatin (GelMA), methacrylated sodium alginate (AlgMA), and methacrylated hyaluronic acid (HAMA).
